(19) United States
(12) Patent Application Publication
(10) Pub. No.: US 2015/0260709 A1
(43) Pub. Date: Sep. 17, 2015

(54) DIAGNOSTIC DEVICE AND DIAGNOSTIC SYSTEM HAVING THE SAME

(71) Applicant: SAMSUNG ELECTRONICS CO., LTD., Suwon-si (KR)

(72) Inventors: Takashi SHIMAYAMA, Seoul (KR); Soo Hong KIM, Suwon-si (KR); Sung Ha PARK, Suwon-si (KR)

(73) Assignee: SAMSUNG ELECTRONICS CO., LTD., Suwon-si (KR)

(21) Appl. No.: 14/644,853

(22) Filed: Mar. 11, 2015

Related U.S. Application Data
(60) Provisional application No. 61/979,307, filed on Apr. 14, 2014.

(30) Foreign Application Priority Data
Mar. 12, 2014 (KR) 10-2014-0028679

(51) Int. Cl. G01N 33/53; B01L 3/00

(52) U.S. Cl. CPC G01N 33/5304; B01L 3/5025; B01L 2300/0636; B01L 2300/16

(57) ABSTRACT

A diagnostic device capable of measuring a concentration of glycated hemoglobin using a simple structure and a diagnostic system having the same are provided. The diagnostic device includes: a first sample supplier including a bond inhibiting substance that inhibits bonding between glycated hemoglobin and a bonding substance bound selectively to the glycated hemoglobin; a second sample supplier separated from the first sample supplier; a first chamber connected to the first sample supplier through a first passage between the first sample supplier and the first chamber; and a second chamber connected to the second sample supplier through a second passage between the second sample supplier and the second chamber.

### CROSS-REFERENCE TO RELATED APPLICATIONS

This application claims priority from Korean Patent Application No. 10-2014-0028679, filed on Mar. 12, 2014 in the Korean Intellectual Property Office, and claims the benefit of U.S. Patent Application No. 61/979,307, filed on Apr. 14, 2014 in the United States Patent and Trademark Office, the disclosures of which are incorporated herein by reference in their entireties.

### BACKGROUND

1. Field

Apparatuses and methods consistent with exemplary embodiments relate to a diagnostic device capable of performing in vitro diagnosis using a small amount of sample and a diagnostic system having the same.

2. Description of the Related Art

In order to perform in vitro diagnosis, an immune test, a clinical chemistry test, and the like are performed on a patient's sample (e.g., blood sample). The immune test and the clinical chemistry test are important to diagnose a patient's state, treat the state, and determine a prognosis thereof.

Such in vitro diagnosis is generally performed in a test room or a laboratory of a hospital. However, an in vitro diagnostic device having a small size may be used in order to perform in vitro diagnosis anywhere.

In addition, in order to rapidly perform in vitro diagnosis under emergency situations, the time taken for in vitro diagnosis should be minimized.

### SUMMARY

Aspects of one or more exemplary embodiments provide a diagnostic device capable of measuring a concentration of glycated hemoglobin using a simple structure and a diagnostic system having the same.

According to an aspect of an exemplary embodiment, there is included a diagnostic device, including: a first sample supplier including a bond inhibiting substance that inhibits bonding between glycated hemoglobin and a bonding substance bound selectively to the glycated hemoglobin; a second sample supplier separated from the first sample supplier; a first chamber connected to the first sample supplier through a first passage between the first sample supplier and the first chamber; and a second chamber connected to the second sample supplier through a second passage between the second sample supplier and the second chamber.

The first sample supplier may include a first sample inlet to receive a sample including a carrier to which the bonding substance is conjugated, and the second sample supplier may include a second sample inlet to receive the sample.

The bonding substance may include at least one of boronic acid, boronic acid derivatives, antibodies, and concanavalin A.

The bond inhibiting substance may include at least one of sorbitol, fructose, xylose, and an acid solution.

The first passage and the second passage may each have a width at which an aggregate of carriers to which the bonding substance is conjugated is unable to pass.

The first passage may have a width of 1 μm to 500 μm, and the second passage may have a width of 1 μm to 500 μm.

The device may further include: a measurement device including an upper plate, a lower plate, and an intermediate plate between the upper plate and the lower plate, wherein the first sample supplier, the second sample supplier, the first passage, the second passage, the first chamber, and the second chamber may be included in the measurement device.

The upper plate and the lower plate may include at least one of a polyethylene film, a very low density polyethylene (VLDPE) film, a linear low density polyethylene (LLDPE) film, a low-density polyethylene (LDPE) film, a medium-density polyethylene (MDPE) film, a high-density polyethylene (HDPE) film, a polypropylene (PP) film, a polyvinyl chloride (PVC) film, a polyvinyl alcohol (PVA) film, a polystyrene (PS) film, and a polyethylene terephthalate (PET) film.

The intermediate plate may include a porous membrane having hydrophobicity.

The device may further include: a third sample supplier including the bond inhibiting substance and separated from the first sample supplier and the second sample supplier; a fourth sample supplier separated from the first sample supplier, the second sample supplier, and the third sample supplier; a third chamber connected to the third sample supplier through a third passage between the third sample supplier and the third chamber; and a fourth chamber connected to the fourth sample supplier through a fourth passage between the fourth sample supplier and the fourth chamber, wherein the fourth sample supplier does not include the bond inhibiting substance.

According to an aspect of another exemplary embodiment, there is provided a diagnostic system, including: a carrier to which a bonding substance bound selectively to glycated hemoglobin is conjugated; and a diagnostic device including: a first sample supplier configured to accommodate a bond inhibiting substance that inhibits the bonding substance from binding to the glycated hemoglobin, a second sample supplier separated from the first sample supplier, a first chamber connected to the first sample supplier through a first passage between the first sample supplier and the first chamber, and a second chamber connected to the second sample supplier through a second passage between the second sample supplier and the second chamber.

The carrier may include at least one of agarose beads, latex beads, sepharose beads, polyethylene glycol beads, glass beads, dextran beads, polystyrene beads, polyvinyl toluene beads, and polymethylmethacrylate beads.

The bonding substance may include at least one of boronic acid, boronic acid derivatives, antibodies, and concanavalin A.

The bond inhibiting substance may include at least one of sorbitol, fructose, xylose, and an acid solution.

The first sample supplier may include the bond inhibiting substance, and the second sample supplier may not include the bond inhibiting substance.

According to an aspect of another exemplary embodiment, there is provided a diagnostic device, including: a first sample supplier configured to receive a sample and including a bond inhibiting substance that inhibits bonding between a target material in the sample and a bonding substance bound selectively to the target material; a second sample supplier configured to receive the sample and separated from the first sample supplier; a first chamber connected to the first sample supplier through a first passage between the first sample supplier and the first chamber; and a second chamber connected to the second sample supplier through a second passage between the second sample supplier and the second chamber.

The bond inhibiting substance may inhibit bonding between glycated hemoglobin in the sample and a bonding substance bound selectively to the glycated hemoglobin.

The second sample supplier may not include the bond inhibiting substance.

The first sample supplier may include a first sample inlet to receive the sample including a carrier to which the bonding substance is conjugated, and the second sample supplier may include a second sample inlet to receive the sample.

The first passage and the second passage may each have a width at which an aggregate of carriers to which the bonding substance is conjugated is unable to pass.

### BRIEF DESCRIPTION OF THE DRAWINGS

These and/or other aspects will become apparent and more readily appreciated from the following description of exemplary embodiments, taken in conjunction with the accompanying drawings of which:

FIG. 1 is a diagram schematically illustrating a bonding reaction between a glycated hemoglobin and a bonding substance carrier;

FIG. 2 is a diagram schematically illustrating an exemplary diagnostic device according to an exemplary embodiment;

FIG. 3 is a diagram schematically illustrating a phenomenon occurring in a first sample supply unit;

FIG. 4 is a diagram schematically illustrating a phenomenon in which hemoglobin, included in a sample, pass a first passage and are introduced into a first chamber;

FIG. 5 is a diagram schematically illustrating a phenomenon occurring in a second sample supply unit;

FIG. 6 is a diagram schematically illustrating a phenomenon in which normal hemoglobin, included in a sample, pass a second passage and are introduced into a second chamber;

FIG. 7 is a graph showing the result obtained by measuring a capture performance of glycated hemoglobin by boronic acid beads;

FIG. 8 is a diagram illustrating an appearance of a diagnostic device according to an exemplary embodiment;

FIG. 9 is a plan view of a housing of the diagnostic device seen from the top according to an exemplary embodiment;

FIG. 10 is a diagram illustrating an appearance of another example of a first sample supply hole and a second sample supply hole;

FIG. 11 is an exploded perspective view of a measurement unit of the diagnostic device according to an exemplary embodiment;

FIG. 12 is a plan view of an intermediate plate of the diagnostic device according to an exemplary embodiment;

FIG. 13 is a cross-sectional side view of the diagnostic device according to an exemplary embodiment;

FIG. 14 is a diagram illustrating an appearance of a diagnostic device, capable of testing several types of samples, according to an exemplary embodiment;

FIG. 15 is a plan view of an intermediate plate included in the diagnostic device of FIG. 14;

FIG. 16 is a diagram illustrating an appearance of a diagnostic system according to an exemplary embodiment;

FIG. 17 is a diagram illustrating an appearance of a test device configured to obtain a diagnostic result using a diagnostic device;

FIG. 18 is a control block diagram of the test device configured to obtain a diagnostic result using a diagnostic device; and

FIG. 19 is a graph showing absorbance obtained by radiating light onto a first chamber and a second chamber.

### DETAILED DESCRIPTION OF EXEMPLARY EMBODIMENTS

Hereinafter, a diagnostic device and a diagnostic system having the same according to exemplary embodiments will be described in detail with reference to the accompanying drawings. Expressions such as “at least one of,” when preceding a list of elements, modify the entire list of elements and do not modify the individual elements of the list.

A diagnostic device according to an exemplary embodiment may, by way of example, be used to measure a concentration of a target material in a sample. In the below description, glycated hemoglobin is exemplarily described as the target material. The glycated hemoglobin is a form of hemoglobin in which hemoglobin molecules of red blood cells carrying oxygen are bound to glucose in blood. A level thereof may be accepted or understood as a blood glucose level during the last two to three months.

In general, hemoglobin in red blood cells of adults may be classified as HbA (90%), HbA1 (7%), or HbF (0.5%). HbA1 may be subdivided into HbA1a, HbA1b, and HbA1c. The value denoted in parentheses refers to a percentage. HbA1c is a type of glycated hemoglobin in which glucose is bound to the N-terminal valine of the hemoglobin. Furthermore, glycated hemoglobin may refer to HbA1c. However, in the current description of exemplary embodiments, glycated hemoglobin does not necessarily refer to only HbA1c but may refer to all or various types of hemoglobin that are bound to glucose.

A quantitative level of the glycated hemoglobin may be represented as a ratio of a concentration of the glycated hemoglobin relative to a concentration of total hemoglobin in blood. In a related art, in order to independently measure the concentration of the total hemoglobin and the concentration of the glycated hemoglobin, step-by-step movement and washing of the sample in a diagnostic device is performed. In order to implement such a process, complex structures such as a plurality of chambers and passages (i.e., channels) are included in the diagnostic device, and a device capable of rotating or vibrating the diagnostic device is utilized.

A diagnostic device according to an exemplary embodiment uses a simple structure without a rotating or vibrating process to measure the concentration of the total hemoglobin and the concentration of the glycated hemoglobin in a sample. A method of measuring the concentration of the glycated hemoglobin includes at least one of an immunoassay, ion-exchange high-performance liquid chromatography, affinity, an enzymatic assay, capillary electrophoresis, and the like. The diagnostic device according to the present exemplary embodiment measures the concentration of the glycated hemoglobin by applying the affinity method.

A sample injected into the diagnostic device according to the present exemplary embodiment may be blood. The sample may be injected into the diagnostic device in a hemolyzed state. For this purpose, the sample may react with a hemolysis reagent. At least one of tris(hydroxymethyl)aminomethane (TRIS), 2-[4-(2-hydroxyethyl)piperazin-1-yl]ethanesulfonic acid (HEPES), piperazine-N,N′-bis(2-ethanesulfonic acid) (PIPES), and 2-[[1,3-dihydroxy-2-(hydroxymethyl)propan-2-yl]amino]ethanesulfonic acid (TES) may be used as the hemolysis reagent. A surfactant may be further added thereto. However, it is understood that one or more other exemplary embodiments are not limited to the hemolysis reagent. In addition to the exemplified hemolysis reagent, any hemolysis reagent that can separate hemoglobin from red blood cells in a blood sample may be used.

The diagnostic device according to the present exemplary embodiment applies the affinity method to measure the concentration of the glycated hemoglobin (as an example of a first target material). In order to apply the affinity method, a bonding substance that is bound selectively to the glycated hemoglobin may be used (although in another exemplary embodiment, a bonding substance that is bound selectively to normal hemoglobin (as an example of a second target material), and not the glycated hemoglobin, may be used).

As the bonding substance that is bound selectively to the glycated hemoglobin, at least one of boronic acid, boronic acid derivatives, concanavalin A, and antibodies may be used. The bonding substance that may be used is conjugated to a carrier. In the description of exemplary embodiments, a conjugate of the bonding substance bound selectively to the glycated hemoglobin and the carrier is referred to as a bonding substance carrier.

In addition, beads may be used as the carrier conjugated to the bonding substance. As a specific example, at least one of agarose beads, latex beads, sepharose beads, polyethylene glycol beads, glass beads, dextran beads, polystyrene beads, polyvinyl toluene beads, and polymethylmethacrylate beads may be used. Hereinafter, the following description of an exemplary embodiment will exemplify beads as the carrier conjugated to the bonding substance.

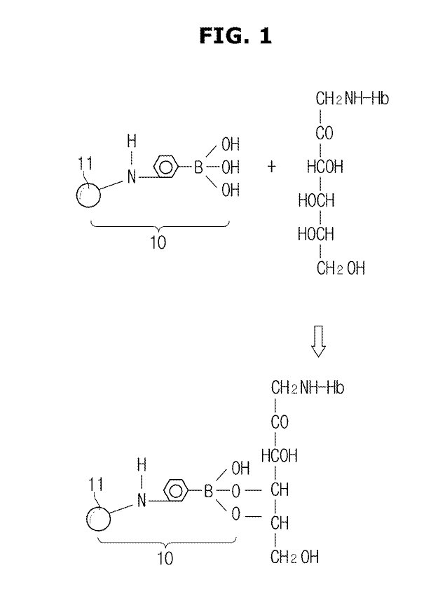

FIG. 1 is a diagram schematically illustrating a bonding reaction between glycated hemoglobin and a bonding substance carrier.

As the bonding substance bound selectively to the glycated hemoglobin, as exemplified in FIG. 1, m-aminophenylboronic acid may be used. When a boronic acid carrier 10 in which boronic acid is conjugated to a carrier 11 meets the glycated hemoglobin, cis-diol groups of a terminus of the glycated hemoglobin react with and bind to OH groups of the boronic acid as illustrated in FIG. 1.

A user puts the bonding substance carrier 10, a hemolysis reagent, and a sample in a single container and mixes the bonding substance carrier 10, the hemolysis reagent, and the sample. This mixed solution may be injected into a diagnostic device. For example, the user may strongly shake the container in which the bonding substance carrier 10, the hemolysis reagent, and the sample are included, about 10 times, and then inject the solution included in the container into the diagnostic device. In the description of exemplary embodiments, the user refers to any person who analyzes the sample using the diagnostic device, such as clinical pathologists, physicians, nurses, and patients.

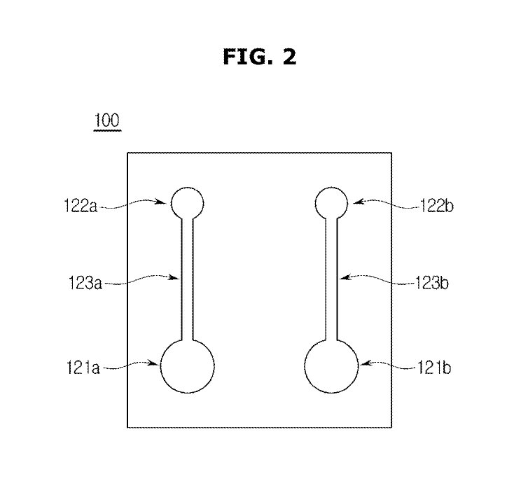

FIG. 2 is a diagram schematically illustrating an exemplary diagnostic device 100 according to an exemplary embodiment.

As illustrated in FIG. 2, a diagnostic device 100 according to an exemplary embodiment includes two sample supply units 121a and 121b (e.g., sample suppliers, sample chambers, sample supply inlets, or sample supply holes), two chambers 122a and 122b corresponding to sample inlets, and two passages 123a and 123b (e.g., channels) connecting the sample supply units 121a and 121b and the two chambers 122a and 122b.

The sample is injected into the two sample supply units 121a and 121b. The sample to be injected is the sample in which the bonding substance carrier 10 and the hemolysis reagent are mixed as described with reference to FIG. 1. The user may drop or inject the sample into the two sample supply units 121a and 121b using an instrument such as a pipet or a dropping pipette.

The first sample supply unit 121a accommodates or includes a bond inhibiting substance that inhibits bonding of the glycated hemoglobin and the bonding substance. Meanwhile, no bond inhibiting substance is accommodated in the second sample supply unit 121b.

For example, the bond inhibiting substance may be at least one of a combination of a saccharide having a large bond constant with boronic acid such as sorbitol, fructose, and xylose, and an acid solution. However, it is understood that one or more other exemplary embodiments are not limited, in terms of the bond inhibiting substance, thereto. In addition to the above example, any substance capable of inhibiting bonding between the glycated hemoglobin and the bonding substance may be used.

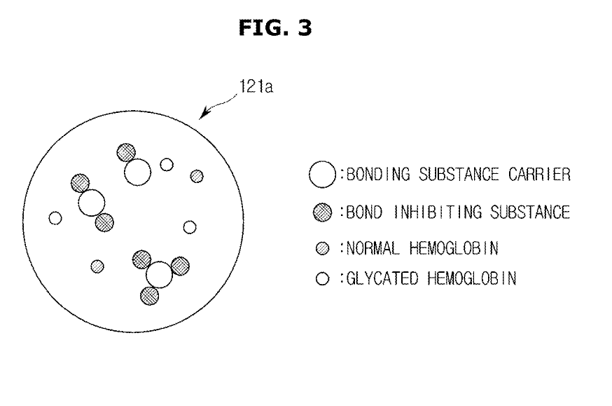

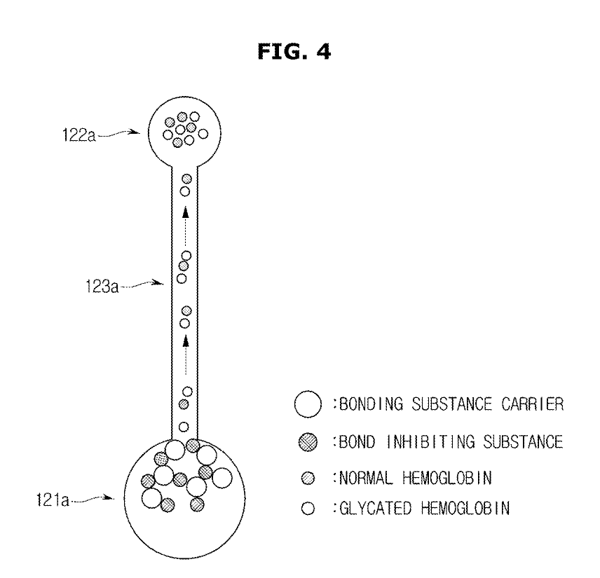

FIG. 3 is a diagram schematically illustrating a phenomenon occurring in a first sample supply unit 121a. FIG. 4 is a diagram schematically illustrating a phenomenon in which hemoglobin included in a sample pass a first passage 123a and are introduced into a first chamber 122a.

As illustrated in FIG. 3, the sample injected into the first sample supply unit 121a includes the glycated hemoglobin (as an example of a first target material), normal hemoglobin (as an example of a second target material), and the bonding substance carrier. Also, some of the bonding substance carrier may be previously bound to the glycated hemoglobin included in the sample before the carrier is injected into the first sample supply unit 121a.

However, since the bond inhibiting substance is accommodated in the first sample supply unit 121a, the bonding substance carrier bound to the glycated hemoglobin is released from the glycated hemoglobin, and a bonding reaction between an unbound bonding substance carrier and the glycated hemoglobin is inhibited.

Meanwhile, since an aggregate, in which bonding substance carriers are collected and grouped together, is unable to pass through the first passage 123a or the second passage 123b, the bonding substance carrier may not be introduced into the first chamber 122a. Therefore, as illustrated in FIG. 4, from the sample injected into the first sample supply unit 121a, only the normal hemoglobin and the glycated hemoglobin may be introduced into the first chamber 122a through the first passage 123a. The concentration of the total hemoglobin may be measured using the first chamber 122a. Measuring the concentration of the total hemoglobin will be described in detail below.

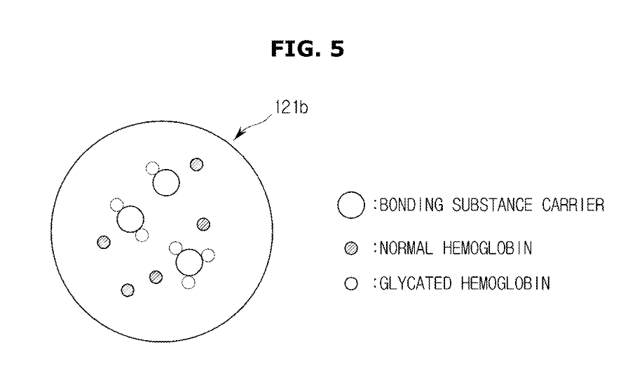

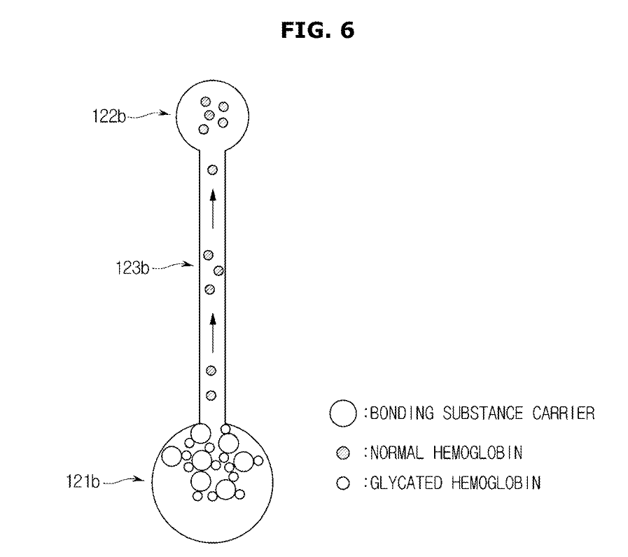

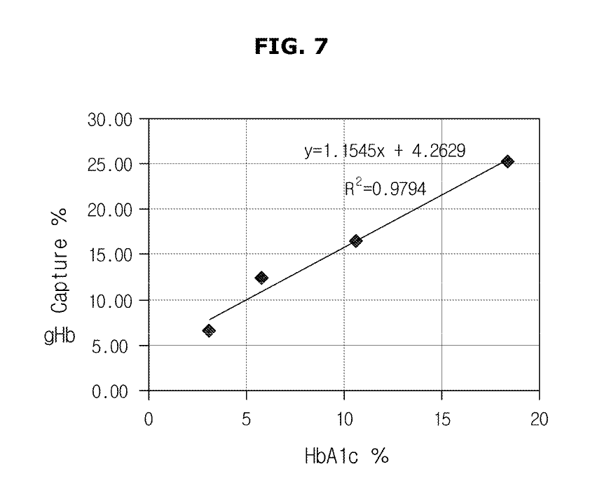

FIG. 5 is a diagram schematically illustrating a phenomenon occurring in a second sample supply unit 121b. FIG. 6 is a diagram schematically illustrating a phenomenon in which normal hemoglobin included in a sample pass through a second passage 123b and are introduced into a second chamber 122b. FIG. 7 is a graph showing the result obtained by measuring a capture performance of glycated hemoglobin by boronic acid beads.

The sample injected into the second sample supply unit 121b is the same as the sample injected into the first sample supply unit 121a. Therefore, the sample injected into the second sample supply unit 121b also includes the glycated hemoglobin, the normal hemoglobin, and the bonding substance carrier. Since no bond inhibiting substance is accommodated in the second sample supply unit 121b, a bonding reaction between the glycated hemoglobin and the bonding substance carrier may occur in the second sample supply unit 121b. Also, as described above, some of the bonding reaction may occur in advance when the hemolysis reagent, the bonding substance carrier, and the sample are mixed before the sample is injected into the second sample supply unit 121b.

As illustrated in FIG. 6, since the normal hemoglobin does not bind to the bonding substance carrier, the normal hemoglobin is introduced into the second chamber 122b through the second passage 123b, but the glycated hemoglobin bound to the bonding substance carrier is unable to pass through the second passage 123b. A width of the second passage 123b may be implemented to be smaller than a diameter of the bonding substance carrier. However, even when the width of the second passage 123b is greater than the diameter of the bonding substance carrier, since the bonding substance carriers exist in a collected and grouped aggregation state in the second sample supply unit 121b, the bonding substance carriers are unable to pass through the second passage 123b.

Through an experiment, the width of the second passage 123b was set in an exemplary embodiment to 300 μm, the diameter of the bonding substance carrier was set to 100 μm, and the second chamber 122b was imaged using a microscope. There was no bonding substance carrier in the second chamber 122b. Accordingly, even when the width of the second passage 123b is several times greater than the diameter of the bonding substance carrier, it may be observed that the normal hemoglobin and the glycated hemoglobin may be separated in the second sample supply unit 121b.

The graph in FIG. 7 shows the result obtained when a sample including HbA1c of a known concentration reacts with boronic acid beads and then a concentration of the glycated hemoglobin captured by the boronic acid beads is measured. As shown in the graph of FIG. 7, as the concentration of HbA1c increases, the concentration of the captured glycated hemoglobin linearly increases. Therefore, the boronic acid beads may capture almost all glycated hemoglobin included in the sample. Accordingly, when the boronic acid bead is used as the bonding substance carrier, it is possible to secure a high reliability of separation of the normal hemoglobin from the glycated hemoglobin.

As described above with reference to FIGS. 2 to 7, when a hemolyzed sample mixed with the bonding substance carrier is injected into the first sample supply unit 121a in which the bond inhibiting substance is accommodated and into the second sample supply unit 121b in which no bond inhibiting substance is accommodated, in the diagnostic device 100, both the glycated hemoglobin and the normal hemoglobin are introduced into the first chamber 122a, and only the normal hemoglobin are introduced into the second chamber 122b. Therefore, it is possible to obtain the concentration of the total hemoglobin included in the sample using the first chamber 122a, and it is possible to obtain the concentration of the normal hemoglobin included in the sample using the second chamber 122b. Then, it is possible to determine the concentration of the glycated hemoglobin from the concentration of the total hemoglobin and the concentration of the normal hemoglobin.

That is, when the diagnostic device 100 is used, it is possible to obtain the concentration of the glycated hemoglobin included in the sample using a simple structure without a vibrating, rotating, or washing process. Hereinafter, a specific structure of the diagnostic device 100 according to an exemplary embodiment will be described.

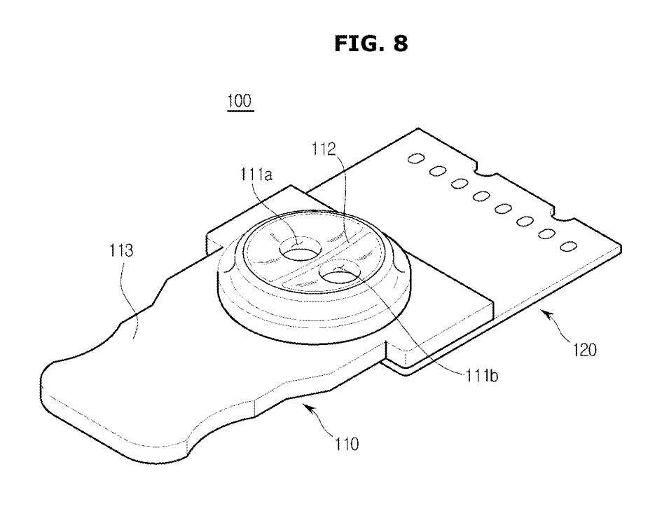

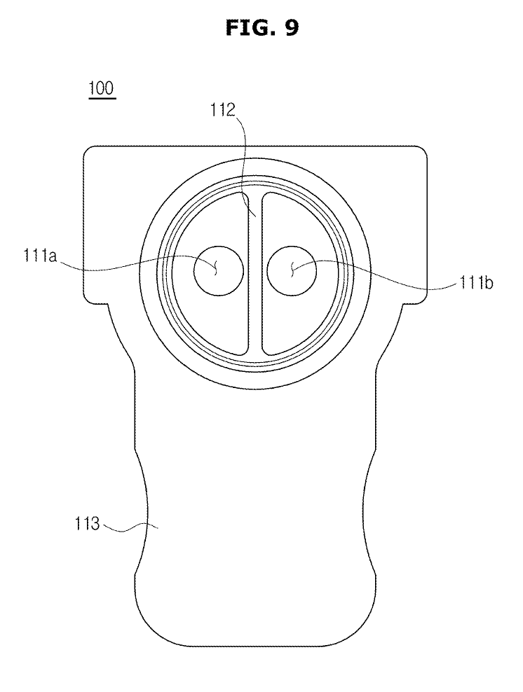

FIG. 8 is a diagram illustrating an appearance of a diagnostic device 100 according to an exemplary embodiment. FIG. 9 is a plan view of a housing of the diagnostic device 100, seen from the top according to an exemplary embodiment.

The diagnostic device 100 according to the present exemplary embodiment illustrated in FIG. 8 may include a housing 110 and a measurement unit 120 (e.g., measurement device) in which structures such as a sample supply unit, a passage, and a chamber are formed (or provided) and reactions for measurement occur.

The housing 110 supports the measurement unit 120 and provides (or includes) a gripper 113 which enables the user to hold the diagnostic device 100. The diagnostic device 100 is advantageous in that it can quickly test the sample anywhere. In particular, in a test of a bio sample taken from a human body, a test performed in a place, for example, a home, a workplace, an outpatient clinic, a hospital room, an emergency room, an operation room, and an intensive care unit, outside a laboratory, may be referred to as a point of care testing (POCT).

The diagnostic device 100 used in the POCT may frequently be carried by the user. Since the diagnostic device 100 includes the gripper 113 that can be easily grasped by the user, the user may stably carry the diagnostic device 100 through the gripper 113.

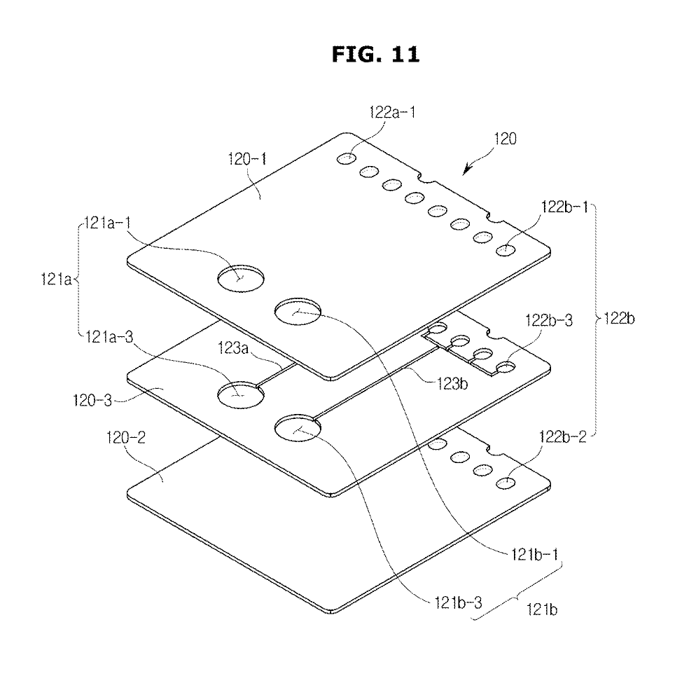

Referring to FIGS. 8 and 9, a first sample supply hole 111a and a second sample supply hole 111b are formed (e.g., included) in the housing 110. The first sample supply hole 111 a and the second sample supply hole 111 b correspond to the first sample supply unit 121a (refer to FIG. 11) and the second sample supply unit 121b (refer to FIG. 11) formed in the measurement unit 120, respectively. Therefore, the sample supplied through the first sample supply hole 111a is injected into the first sample supply unit 121a and the sample supplied through the second sample supply hole 111 b is injected into the second sample supply unit 121 b.

The housing 110 may be formed of (e.g., include) a material that is easy to form and has a chemically and biologically inert state. Various materials, for example, an acryl such as polymethylmethacrylate (PMMA), a polysiloxane such as polydimethylsiloxane (PDMS), polycarbonate (PC), a polyethylene such as linear low density polyethylene (LLDPE), a low-density polyethylene (LDPE), a medium-density polyethylene (MDPE), and a high-density polyethylene (HDPE), plastic materials such as polyvinyl alcohol, a very low density polyethylene (VLDPE), a polypropylene (PP), acrylonitrile-butadiene-styrene (ABS), and a cycloolefin copolymer (COC), glass, mica, silica, and a semiconductor wafer, may be used as the material of the housing 110. However, the material of the housing 110 is not limited to the above examples.

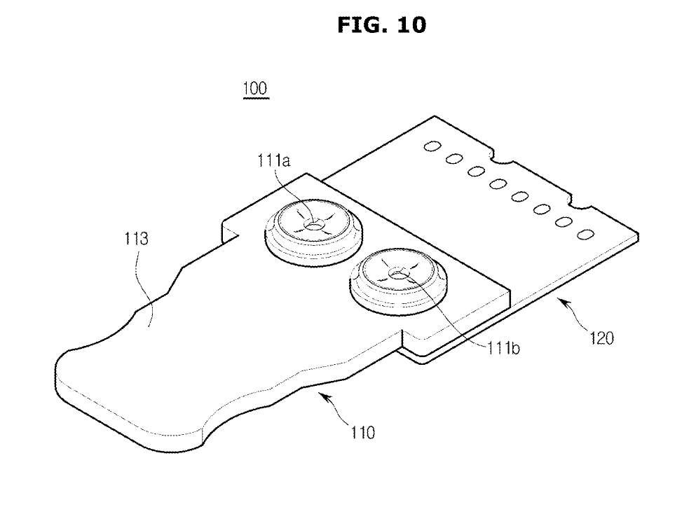

FIG. 10 is a diagram illustrating an appearance of another example of a first sample supply hole 111 a and a second sample supply hole 111b.

As will be described below, the first sample supply unit 121a and the second sample supply unit 121b may be physically separated such that reactions or phenomena occurring therein do not influence each other. Therefore, the first sample supply hole 111a and the second sample supply hole 111b corresponding to the first sample supply unit 121a and the second sample supply unit 121b, respectively, may also be separately formed or provided, and may be separated by a partition wall 112 as illustrated in FIGS. 8 and 9. As illustrated in FIG. 10, the first sample supply hole 111a and the second sample supply hole 111b may also be formed or provided as separately independent structures in one or more other exemplary embodiments.

However, it is understood that the structure of the diagnostic device 100 is not limited to the examples of FIGS. 8 to 10. For example, a shape or a position thereof is not limited, as long as the first sample supply hole 111a and the second sample supply hole 111b are separately formed. According to still another exemplary embodiment, a single sample supply hole may be provided, and the first sample supply unit 121a and the second sample supply unit 121b may be fluidly connected to the single sample supply hole and/or may be provided in a same chamber with a partition wall therebetween.

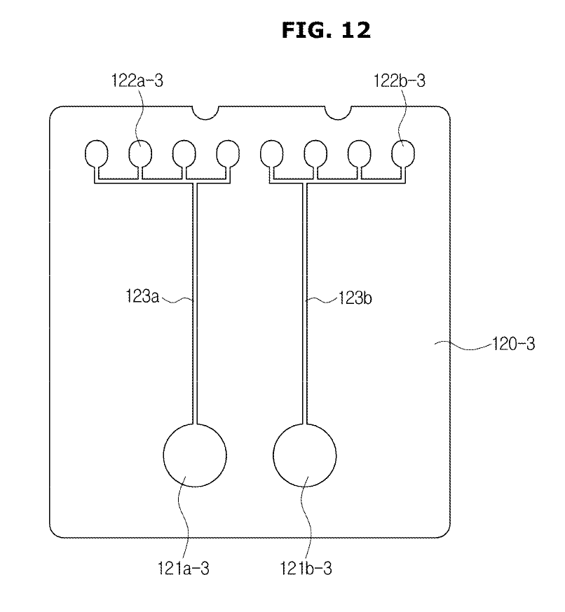

FIG. 11 is an exploded perspective view of a measurement unit 120 of a diagnostic device 100 according to an exemplary embodiment. FIG. 12 is a plan view of an intermediate plate 120-3 of a diagnostic device 100 according to an exemplary embodiment.

As illustrated in FIG. 11, the measurement unit 120 may be formed of (e.g., include) a structure in which three plates 120-1, 120-2, and 120-3 are bound. The three plates may include an upper plate 120-1, a lower plate 120-2, and an intermediate plate 120-3. The upper plate 120-1 and the lower plate 120-2 may protect the sample moving to the chambers 122a and 122b from external light by, for example, printed light-shielding ink.

The upper plate 120-1 and the lower plate 120-2 may be formed of a film. The film used to form the upper plate 120-1 and the lower plate 120-2 may be at least one of a polyethylene film such as VLDPE, LLDPE, LDPE, MDPE, and HDPE, a PP film, a polyvinyl chloride (PVC) film, a polyvinyl alcohol (PVA) film, a polystyrene (PS) film, and a polyethylene terephthalate (PET) film.

The intermediate plate 120-3 is formed of a porous sheet such as cellulose and may directly serve as a vent. The porous sheet is made of (e.g., includes) a material having hydrophobicity or a hydrophobic treatment is performed on the porous sheet such that movement of the sample may not be influenced.

Structures such as the sample supply units 121a and 121b, the passages 123a and 123b, and the chambers 122a and 122b are formed (e.g., included) in the measurement unit 120. When the measurement unit 120 is formed of a three-layer structure as illustrated in FIG. 11, each structure may be completed when the upper plate 120-1, the lower plate 120-2, and the intermediate plate 120-3 are stacked.

For example, a first upper plate hole 121a-1 of the first sample supply unit 121a and a second upper plate hole 121b-1 of the second sample supply unit 121 b are formed in the upper plate 120-1, and a first chamber window 122a-1 of the first chamber 122a and a second chamber window 122b-1 of the second chamber 122b may be processed to become transparent.

In addition, in the lower plate 120-2, a first chamber window 122a-2 of the first chamber 122a and a second chamber window 122b-2 of the second chamber 122b may be processed to become transparent. The process of developing the transparency of the first chamber windows 122a-1 and 122a-2 and the second chamber windows 122b-1 and 122b-2 formed in the upper plate 120-1 and the lower plate 120-2 may be performed to measure an optical property of the substance introduced into the first chamber 122a and the second chamber 122b.

As illustrated in FIG. 12, in the intermediate plate 120-3, a first intermediate plate hole 121a-3 of the first sample supply unit 121a and a second intermediate plate hole 121b-3 of the second sample supply unit 121b are formed, and a first chamber hole 122a-3 of the first chamber 122a and a second chamber hole 122b-3 of the second chamber 122b are formed.

While the examples of FIGS. 11 and 12 illustrate four first chambers 122a and four second chambers 122b, it is understood that one or more other exemplary embodiments are not limited thereto. A number other than four, such as one first chamber 122a and one second chamber 122b may be provided.

When the upper plate 120-1, the intermediate plate 120-3, and the lower plate 120-2 are bound, the first upper plate hole 121a-1, the first intermediate plate hole 121a-3, and the lower plate 120-2 constitute the first sample supply unit 121a. Furthermore, in this case, the second upper plate hole 121 b-1, the second intermediate plate hole 121b-3, and the lower plate 120-2 constitute the second sample supply unit 121b. In addition, the first chamber window 122a-1 of the upper plate 120-1, the first chamber window 122a-2 of the lower plate 120-2, and the first chamber hole 122a-3 of the intermediate plate 120-3 constitute the first chamber 122a. Furthermore, the second chamber window 122b-1 of the upper plate 120-1, the second chamber window 122b-2 of the lower plate 120-2, and the second chamber hole 122b-3 of the intermediate plate 120-3 constitute the second chamber 122b.

An adhesive may be used to bind the upper plate 120-1, the intermediate plate 120-3, and the lower plate 120-2. For example, a pressure sensitive adhesive (PSA) may be used. The PSA will be described below.

Also, the first passage 123a connecting the first sample supply unit 121a and the first chamber 122a and the second passage 123b connecting the second sample supply unit 121b and the second chamber 122b are formed in the intermediate plate 120-3.

The first passage 123a and the second passage 123b may be formed (e.g., provided) to have a width of 1 μm to 500 μm. The sample injected into the first sample supply unit 121a and the second sample supply unit 121b may move to the first chamber 122a and the second chamber 122b due to a capillary force even when a separate driving force such as rotary power is not provided. However, the above-described widths of the passages 123a and 123b are only an example that can be applied to the diagnostic device 100 and it is understood that one or more other exemplary embodiments are not limited thereto.

Meanwhile, in the above example, while the structures such as the chamber or the passage constituting the diagnostic device 100 are formed in the intermediate plate 120-3, it is understood that one or more other exemplary embodiments are not limited thereto. For example, the structures may be formed in the upper plate 120-1 or the lower plate 120-2 as intaglio structures.

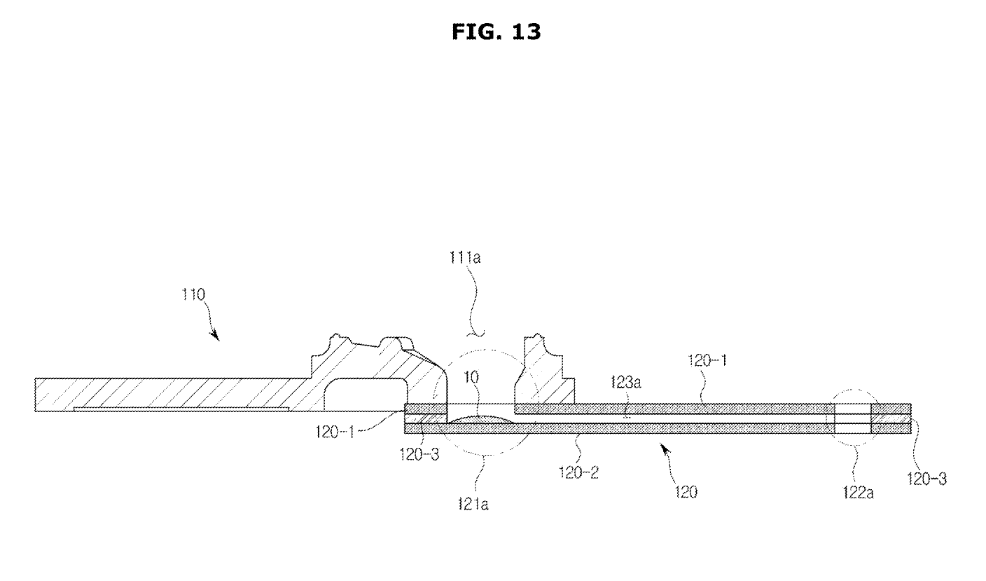

FIG. 13 is a cross-sectional side view of the diagnostic device 100 according to an exemplary embodiment. The cross-sectional side view of FIG. 13 is seen from the first sample supply unit 121a side.

The measurement unit 120 is bound to a bottom of the sample supply holes 111a and 111b of the housing 110 to form the single diagnostic device 100. In this case, the first sample supply hole 111a of the housing 110 may be connected to the first sample supply unit 121a of the measurement unit 120, and the second sample supply hole 111b of the housing 110 may be connected to the second sample supply unit 121b of the measurement unit 120.

For example, when the housing 110 and the measurement unit 120 are bound, the PSA may be used. The PSA has properties in that the PSA can be adhered to an adherend in a short time with a low pressure of about a finger pressure at room temperature, no cohesive failure occurs during detachment, and no residue remains on a surface of the adherend.

However, bonding the housing 110 and the measurement unit 120 may not be or may not only be performed by the PSA in one or more other exemplary embodiments. For example, the bonding may be performed by other double-sided adhesives rather than the PSA, or may be performed in a manner in which a protrusion is inserted into a groove.

Meanwhile, the bond inhibiting substance 10 is accommodated in the first sample supply unit 121a. As an example of accommodating the bond inhibiting substance 10 in the first sample supply unit 121a, the bond inhibiting substance 10 may be applied onto the lower plate 120-2 that is a bottom of the first sample supply unit 121a and then dried, as illustrated in FIG. 12. When there is a membrane in the first sample supply unit 121a, the substance may also be applied onto the membrane.

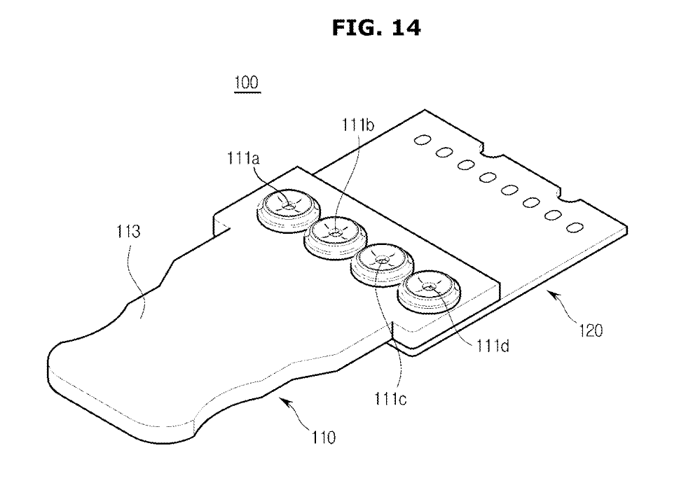

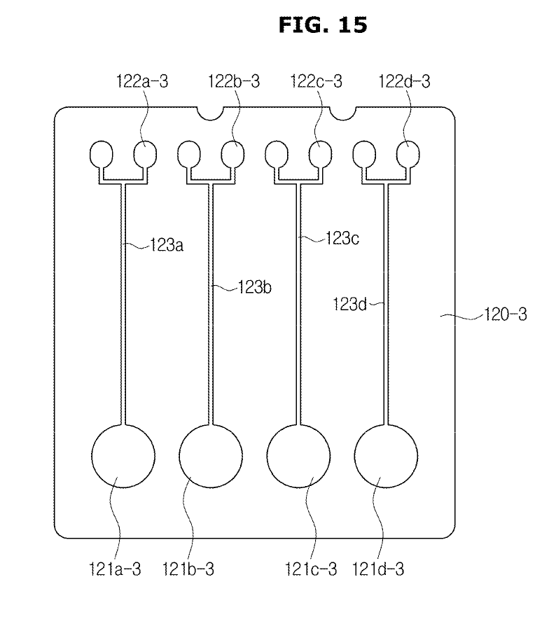

FIG. 14 is a diagram illustrating an appearance of a diagnostic device 100 capable of testing several types of samples, according to an exemplary embodiment. FIG. 15 is a plan view of an intermediate plate 120-3 included in the diagnostic device 100 of FIG. 14.

As illustrated in FIG. 14, the diagnostic device 100 may include four sample supply holes 111a, 111b, 111c, and 111d. As illustrated in FIG. 15, four intermediate plate holes 121a-3, 121b-3, 121c-3, and 121d-3 corresponding to the four sample supply holes 111a, 111b, 111c, and 111d, respectively, may be provided in the intermediate plate 120-3. Also, from the four intermediate plate holes 121a-3, 121b-3, 121c-3, and 121d-3, the first intermediate plate hole 121a-3 is connected to the first chamber hole 122a-3 through the first passage 123a, the second intermediate plate hole 121b-3 is connected to the second chamber hole 122b-3 through the second passage 123b, the third intermediate plate hole 121c-3 is connected to a third chamber hole 122c-3 through a third passage 123c, and the fourth intermediate plate hole 121d-3 is connected to a fourth chamber hole 122d-3 through a fourth passage 123d.

From the four sample supply holes 111a, 111b, 111c, and 111d, the first sample supply hole 111a and the second sample supply hole 111b may serve as a set, and the same sample may be dropped thereinto. The third sample supply hole 111c and the fourth sample supply hole 111d may serve as a set and a sample different from the sample dropped into the first sample supply hole 111a and the second sample supply hole 111b may be dropped thereinto.

For example, a blood sample of a patient A may be dropped into the first sample supply hole 111a and the second sample supply hole 111b, and a blood sample of a patient B may be dropped into the third sample supply hole 111c and the fourth sample supply hole 111d. In this case, diabetes tests for two patients may be performed using the single diagnostic device 100.

As another example, a blood sample X of a certain patient may be dropped into the first sample supply hole 111a and the second sample supply hole 111b, and a blood sample Y of the same patient may be dropped into the third sample supply hole 111c and the fourth sample supply hole 111d. In this case, the result of simultaneously testing the blood sample of the same patient twice may be obtained using the single diagnostic device 100. Accordingly, it is possible to improve the reliability of the diabetes test.

However, it is understood that one or more other exemplary embodiments are not limited to the examples of FIGS. 14 and 15. For example, in one or more other exemplary embodiments, the number of sample supply units may be adjusted as necessary or desired such that a plurality of test results may be obtained in the single diagnostic device 100.

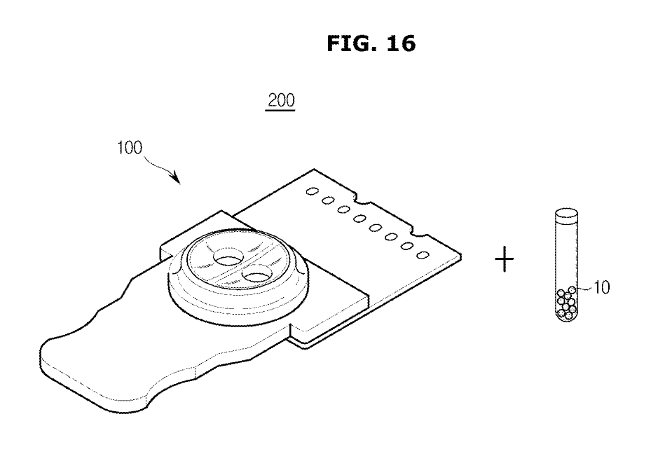

FIG. 16 is a diagram illustrating an appearance of a diagnostic system 200 according to an exemplary embodiment.

As illustrated in, FIG. 16, a diagnostic system 200 according to an exemplary embodiment may include the diagnostic device 100 and the bonding substance carrier 10 according to the above-described exemplary embodiment. Descriptions of the diagnostic device 100 included in the diagnostic system 200 is the same as or similar to those of FIGS. 2 to 15. As described in FIG. 1, in the bonding substance carrier 10, the bonding substance bound selectively to the glycated hemoglobin is conjugated to the carrier 11. As the carrier 11, at least one of agarose beads, latex beads, sepharose beads, polyethylene glycol beads, glass beads, dextran beads, polystyrene beads, polyvinyl toluene beads, and polymethylmethacrylate beads may be used.

In addition, as the bonding substance, at least one of boronic acid, boronic acid derivatives, concanavalin A, and antibodies may be used.

However, the carrier 11 and the bonding substance are only examples that can be applied to the diagnostic system 200. Carriers or bonding substances other than the above examples may also be used.

In addition, the diagnostic system 200 may further include a hemolysis reagent that separates hemoglobin from red blood cells of the blood sample. The hemolysis reagent may be separately provided from the bonding substance carrier 10, or may be provided in a single reagent along with the bonding substance carrier 10.

The user of the diagnostic system 200 takes a blood sample, and mixes the sample with the hemolysis reagent and the bonding substance carrier 10. The mixed blood sample may be immediately dropped into the first sample supply hole 111a and the second sample supply hole 111b of the diagnostic device 100.

Since the bond inhibiting substance is accommodated in the first sample supply unit 121a, a bonding reaction between the glycated hemoglobin and the bonding substance carrier is inhibited, or the glycated hemoglobin and the bonding substance, which have been bound, are separated.

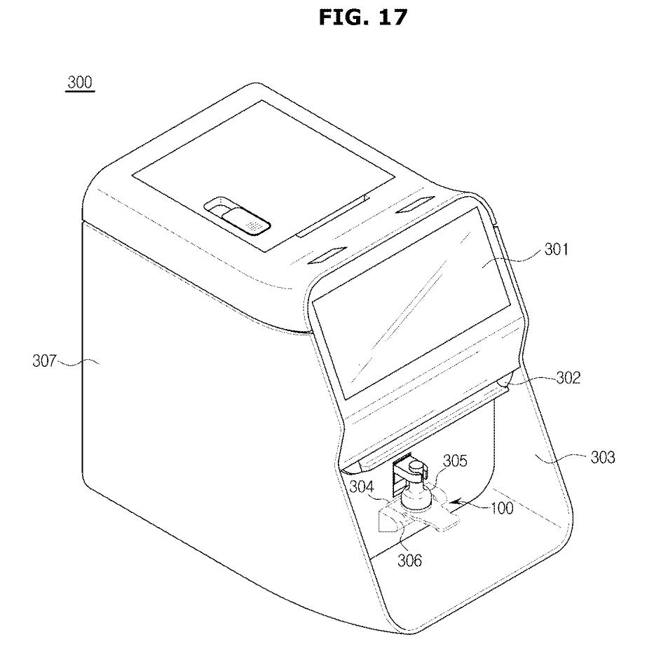

On the other hand, since no bond inhibiting substance is accommodated in the second sample supply unit 121b, a bonding reaction between the glycated hemoglobin and the bonding substance carrier occurs. Processes subsequent thereto will be described with reference to FIGS. 17 to 19.

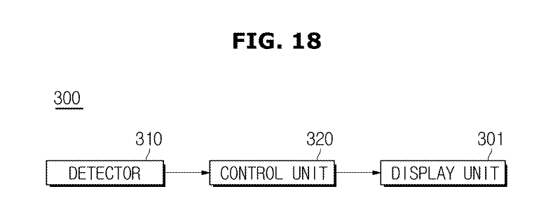

FIG. 17 is a diagram illustrating an appearance of a test device 300 configured to obtain a diagnostic result using a diagnostic device 100, according to an exemplary embodiment. FIG. 18 is a control block diagram of the test device 300 configured to obtain a diagnostic result using a diagnostic device 100, according to an exemplary embodiment.

As illustrated in FIG. 17, a test device 300 includes a mounting unit 303 (e.g., mounting area or mounting structure) that is a space in which the diagnostic device 100 is mounted. When a door 302 of the mounting unit 303 is opened by, for example, sliding upward, the diagnostic device 100 may be mounted on the test device 300. As a specific example, the measurement unit 120 of the diagnostic device 100 may be inserted into a predetermined insertion groove 304 provided in the mounting unit 303.

The measurement unit 120 is inserted into a main body 307. The housing 110 is exposed to the outside of the test device 300 and may be supported by a support 306. When mounting of the diagnostic device 100 is completed and the door 302 is closed, a plunger 305 presses the sample supply holes 111a and 111b to promote introduction of the sample into the measurement unit 120.

As described above, since no bonding reaction between the glycated hemoglobin and the bonding substance carrier occurs in the first sample supply unit 121a, all of the glycated hemoglobin and the normal hemoglobin included in the sample are introduced into the first chamber 122a through the first passage 123a.

Also, since the bonding reaction between the glycated hemoglobin and the bonding substance carrier occurs in the second sample supply unit 121b and the bonding substance carrier is unable to pass through a membrane 130 or the second passage 123b, the normal hemoglobin are introduced into the second chamber 122b and the glycated hemoglobin bonding with the bonding substance carrier are not introduced into the second chamber 122b.

As illustrated in FIG. 18, the test device 300 includes a detector 310 configured to detect properties of a substance accommodated in the first chamber 122a and the second chamber 122b of the diagnostic device 100, a control unit 320 (e.g., controller) configured to obtain a diagnostic result from the detected properties, and a display unit 301 (e.g., display) configured to display the obtained diagnostic result.

The detector 310 may include a light emitting unit (e.g., light emitter) configured to radiate light having a specific wavelength and a light receiving unit (e.g., light receiver or light detector) configured to detect light. The detector 310 may be included inside the main body 307.

As a specific example, the light emitting unit of the detector 310 radiates light having a specific wavelength onto the first chamber 122a and the second chamber 122b, and the light receiving unit detects light transmitting through the first chamber 122a and the second chamber 122b or light reflected from the first chamber 122a and the second chamber 122b. When the light emitting unit and the light receiving unit are disposed at the same side with respect to the measurement unit 120, the reflected light is detected, and when the units are disposed at opposite sides, the transmitted light is detected.

Here, the light having a specific wavelength radiated onto the first chamber 122a and the second chamber 122b may be light having a wavelength that can measure an optical property of the hemoglobin. For example, the light may have a wavelength of a 400 nm band, and more specifically, the light may have a wavelength of 405 nm or 415 nm.

The control unit 320 obtains the optical property from a signal output from the light receiving unit of the detector 310, and may calculate the concentration of the total hemoglobin and the concentration of the normal hemoglobin in the sample based on the optical property. The obtained optical property may include at least one of absorbance, reflectivity, and permeability.

For example, a calibration curve representing a relation between absorbance and the concentration of the hemoglobin may be stored in advance, and the obtained absorbance may be applied to the calibration curve to calculate the concentration of the hemoglobin.

However, as will be described below, since a ratio of the glycated hemoglobin may be obtained using a ratio of the absorbance, calculating an actual concentration using the calibration curve may be omitted.

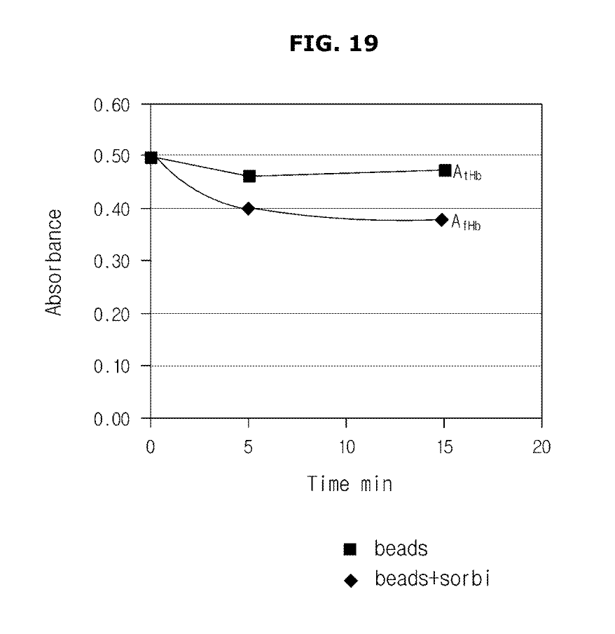

FIG. 19 is a graph showing absorbance obtained by radiating light onto a first chamber 122a and a second chamber 122b according to an exemplary embodiment.

When the detector 310 detects light transmitting through the first chamber 122a, the control unit 320 obtains an absorbance (AtHb) representing the total hemoglobin from an output signal of the detector 310 as illustrated in FIG. 19. When the detector 310 detects light transmitted through the second chamber 122b, the control unit 320 may obtain an absorbance (AfHb) representing the normal hemoglobin from an output signal of the detector 310 as illustrated in FIG. 19.

Then, the control unit 320 may calculate the ratio of the glycated hemoglobin using the following [Equation 1]:

gHb%=[AtHb−AfHb]/AtHb×100 [Equation 1]

Here, gHb% denotes a ratio of glycated hemoglobin.

In addition, the ratio of the glycated hemoglobin calculated by the control unit 320 or the diagnostic result based on the ratio may be displayed through the display unit 301.

According to the diagnostic device 100 and the diagnostic system 200 having the same described above, the concentration of the glycated hemoglobin is measured using a simple structure of the diagnostic device 100. Therefore, it is possible to rapidly perform the diabetes test at a low cost.

In the diagnostic device 100 and the diagnostic system 200 having the same according to an exemplary embodiment, the concentration of the glycated hemoglobin is measured using a simple structure. Therefore, it is possible to rapidly perform the diabetes test at a low cost.

While not restricted thereto, an exemplary embodiment of the control unit 320 can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, the control unit 320 can include circuitry, a processor, a microprocessor, etc., and may execute a computer program stored in a computer-readable medium.

Although a few exemplary embodiments have been shown and described, it would be appreciated by those skilled in the art that changes may be made in these exemplary embodiments without departing from the principles and spirit of the invention, the scope of which is defined in the claims and their equivalents.

What is claimed is:

1. A diagnostic device, comprising:
a first sample supplier comprising a bond inhibiting substance that inhibits bonding between glycated hemoglobin and a bonding substance bound selectively to the glycated hemoglobin;
a second sample supplier separated from the first sample supplier;
a first chamber connected to the first sample supplier through a first passage between the first sample supplier and the first chamber; and
a second chamber connected to the second sample supplier through a second passage between the second sample supplier and the second chamber.

2. The device according to claim 1, wherein:
the first sample supplier comprises a first sample inlet to receive a sample including a carrier to which the bonding substance is conjugated; and
the second sample supplier comprises a second sample inlet to receive the sample.

3. The device according to claim 1, wherein the bonding substance comprises at least one of boronic acid, boronic acid derivatives, antibodies, and concanavalin A.

4. The device according to claim 1, wherein the bond inhibiting substance comprises at least one of sorbitol, fructose, xylose, and an acid solution.

5. The device according to claim 2, wherein the first passage and the second passage each have a width at which an aggregate of carriers to which the bonding substance is conjugated is unable to pass.

6. The device according to claim 2, wherein the first passage has a width of 1 μm to 500 μm, and the second passage has a width of 1 μm to 500 μm.

7. The device according to claim 1, further comprising:
a measurement device comprising an upper plate, a lower plate, and an intermediate plate between the upper plate and the lower plate, wherein the first sample supplier, the second sample supplier, the first passage, the second passage, the first chamber, and the second chamber are comprised in the measurement device.

8. The device according to claim 7, wherein the upper plate and the lower plate comprise at least one of a polyethylene film, a very low density polyethylene (VLDPE) film, a linear low density polyethylene (LLDPE) film, a low-density polyethylene (LDPE) film, a medium-density polyethylene (MDPE) film, a high-density polyethylene (HDPE) film, a polypropylene (PP) film, a polyvinyl chloride (PVC) film, a polyvinyl alcohol (PVA) film, a polystyrene (PS) film, and a polyethylene terephthalate (PET) film.

9. The device according to claim 7, wherein the intermediate plate comprises a porous membrane having hydrophobicity.

10. The device according to claim 1, further comprising:
a third sample supplier comprising the bond inhibiting substance and separated from the first sample supplier and the second sample supplier;
a fourth sample supplier separated from the first sample supplier, the second sample supplier, and the third sample supplier;
a third chamber connected to the third sample supplier through a third passage between the third sample supplier and the third chamber; and
a fourth chamber connected to the fourth sample supplier through a fourth passage between the fourth sample supplier and the fourth chamber,
wherein the fourth sample supplier does not include the bond inhibiting substance.

11. A diagnostic system, comprising:
a carrier to which a bonding substance bound selectively to glycated hemoglobin is conjugated; and
a diagnostic device comprising:
a first sample supplier configured to accommodate a bond inhibiting substance that inhibits the bonding substance from binding to the glycated hemoglobin,
a second sample supplier separated from the first sample supplier,
a first chamber connected to the first sample supplier through a first passage between the first sample supplier and the first chamber, and
a second chamber connected to the second sample supplier through a second passage between the second sample supplier and the second chamber.

12. The system according to claim 11, wherein the carrier comprises at least one of agarose beads, latex beads, sepharose beads, polyethylene glycol beads, glass beads, dextran beads, polystyrene beads, polyvinyl toluene beads, and polymethylmethacrylate beads.

13. The system according to claim 11, wherein the bonding substance comprises at least one of boronic acid, boronic acid derivatives, antibodies, and concanavalin A.

14. The system according to claim 11, wherein the bond inhibiting substance comprises at least one of sorbitol, fructose, xylose, and an acid solution.

15. The system according to claim 11, wherein the first sample supplier comprises the bond inhibiting substance, and the second sample supplier does not include the bond inhibiting substance.

16. A diagnostic device, comprising:
a first sample supplier configured to receive a sample and comprising a bond inhibiting substance that inhibits bonding between a target material in the sample and a bonding substance bound selectively to the target material;
a second sample supplier configured to receive the sample and separated from the first sample supplier;
a first chamber connected to the first sample supplier through a first passage between the first sample supplier and the first chamber; and
a second chamber connected to the second sample supplier through a second passage between the second sample supplier and the second chamber.

17. The device according to claim 16, wherein the bond inhibiting substance inhibits bonding between glycated hemoglobin in the sample and a bonding substance bound selectively to the glycated hemoglobin.

18. The device according to claim 16, wherein the second sample supplier does not include the bond inhibiting substance.

19. The device according to claim 16, wherein:
the first sample supplier comprises a first sample inlet to receive the sample including a carrier to which the bonding substance is conjugated; and
the second sample supplier comprises a second sample inlet to receive the sample.

20. The device according to claim 19, wherein the first passage and the second passage each have a width at which an aggregate of carriers to which the bonding substance is conjugated is unable to pass.
